(19) United States
(12) Patent Application Publication
(10) Pub. No.: US 2023/0372594 A1
(43) Pub. Date: Nov. 23, 2023

(54) SINGLE LUMEN HYBRID CONNECTION TO LEGACY SYSTEM

(71) Applicant: Stavro Medical, Inc., Martinez, CA (US)

(72) Inventor: Lakhmir Singh Chawla, San Diego, CA (US)

(73) Assignee: Stavro Medical, Inc., Martinez, CA (US)

(21) Appl. No.: 18/197,907

(22) Filed: May 16, 2023

Related U.S. Application Data
(60) Provisional application No. 63/343,194, filed on May 18, 2022.

(51) Int. Cl. A61M 1/36; A61M 1/34; A61M 1/16

(52) U.S. Cl. CPC A61M 1/3601; A61M 1/3413; A61M 1/16; A61M 2205/3334

(57) ABSTRACT

The disclosure provides a blood treatment method, comprising: (a) conveying a volume of blood via a first conduit from a vascular access of a patient to a blood chamber at a first flow rate, the first conduit having only a single lumen; (b) conveying the blood from the blood chamber through a filtration device at a second flow rate, wherein the filtration device is part of a legacy system, to perform an extracorporeal treatment on the blood and returning the treated blood to the blood chamber; and (c) returning the blood from the blood chamber to the vascular access of the patient at a third flow rate via the first conduit, wherein the second flow rate is decoupled from, and independent of both the first and third flow rates.

### CROSS-REFERENCES TO RELATED APPLICATIONS

This application claims priority to U.S. Provisional Patent Application No. 63/343,194, filed May 18, 2022, the contents of which are hereby incorporated by reference in its entirety for all purposes.

### BACKGROUND

In extracorporeal blood treatments, blood from a patient (e.g., human or animal) is withdrawn for treatment processing, and the processed blood is subsequently returned to the patient. Conventional extracorporeal blood treatment methods include, but are not limited to, apheresis, plasmapheresis, hemoperfusion (HPF), and renal replacement therapies (RRT), such as hemodialysis (HD), hemofiltration (HF), and hemodiafiltration (HDF) herein after all may be referred to as treatments or blood ‘filtration’ and the devices themselves as ‘filters’. Blood-based RRT systems generally require access to the patient's vascular stream. In conventional RRT systems, sufficient clearance of waste molecules and/or fluids from the processed blood requires a certain blood flow rate through the treatment module.

To accommodate the required blood flow rate for treatment, conventional RRT systems typically require a pair of lumens or needles connected to the patient's blood stream. One of the lumens/needles pulls blood from the patient while the other lumen/needle returns processed blood to the patient, thereby enabling the minimum blood flow required for adequate treatment. For example, conventional RRT systems employ a dual-lumen catheter, an arterio-venous graft, or a matured arterio-venous fistula, all of which require maintenance to assure patency and may be associated with potential complications. Higher clearance levels may require even higher blood flow rates, thereby necessitating larger bores for the lumens/needles withdrawing blood from and returning blood to the patient.

What is needed in the art is a hybrid system that allows a single lumen connection to a legacy system, such as those legacy systems employing a dual-lumen catheter. The present disclosure satisfies this and offers other advantages as well.

### BRIEF SUMMARY

The present disclosure provides a system that allows a single lumen connection to a legacy system, such as those legacy systems employing a dual-lumen catheter. As such, in one embodiment, the present disclosure provides a blood treatment method, comprising:
(a) conveying a volume of blood via a first conduit from a vascular access of a patient to a blood chamber at a first flow rate, the first conduit having only a single lumen;
(b) conveying the blood from the blood chamber through a filtration device at a second flow rate, wherein the filtration device is part of a legacy system, to perform an extracorporeal treatment on the blood and returning the treated blood to the blood chamber; and
(c) returning the blood from the blood chamber to the vascular access of the patient at a third flow rate via the first conduit, wherein the second flow rate is decoupled from, and independent of both the first and third flow rates.

In another embodiment, the present disclosure provides a blood treatment system, the system comprising:
a reservoir for holding a batch of blood from a patient;
a first conduit for conveying blood from a vascular access of the patient during a first stage and for returning treated blood to the vascular access during a third stage, the first conduit having only a single lumen;
a legacy system for performing extracorporeal treatment on blood passing therethrough;
a recirculating blood processing loop connecting the reservoir to a legacy filter and an optional second filter device;
a blood pump for conveying blood in the recirculating blood processing loop; and
a controller configured to control the blood pump to repeatedly recirculate blood from the reservoir through the legacy filter during a second stage between the first and third stages.

In certain aspects, the optional second filter device such as a legacy second filter device is present.

These and other aspects, objects and embodiments will become more apparent when read with detailed description and figures that follow.

### BRIEF DESCRIPTION OF THE DRAWINGS

FIG. 1A is a simplified schematic diagram of the generalized blood treatment system with a single-line vascular access, according to one or more embodiments of the disclosed subject matter.

FIG. 1B is a simplified schematic diagram of a generalized blood treatment system employing batch processing, according to one or more embodiments of the disclosed subject matter.

FIG. 2A is a process flow diagram for a generalized blood treatment method employing batch processing, according to one or more embodiments of the disclosed subject matter.

FIG. 2B is a map illustrating relative timing of various operations in a blood treatment method, according to one or more embodiments of the disclosed subject matter.

FIG. 3A is a simplified schematic diagram of a blood treatment system employing serial processing of multiple blood batches, according to one or more embodiments of the disclosed subject matter.

FIG. 3B is a simplified schematic diagram of a blood treatment system employing parallel processing of multiple blood batches, according to one or more embodiments of the disclosed subject matter.

### DETAILED DESCRIPTION

Extracorporeal blood treatment systems and methods according to the present disclosure employ batch processing of blood (or body fluid) to allow decoupling of the blood flow rate during treatment processing from the blood flow rates used to withdraw/infuse blood from/to the vascular system of a patient (e.g., human or animal). The decoupling of blood flow rates allows for higher blood flow rates during blood treatment to achieve improved clearance, while also allowing for lower blood flow rates to/from the patient, thereby reducing access size (e.g., needle or catheter size) and/or number (e.g. withdraw and infusion ports or access). In certain aspects, the disclosure provides a hybrid system that allows a single lumen connector to a legacy blood processing system, such as those legacy systems that employ a dual-lumen catheter.

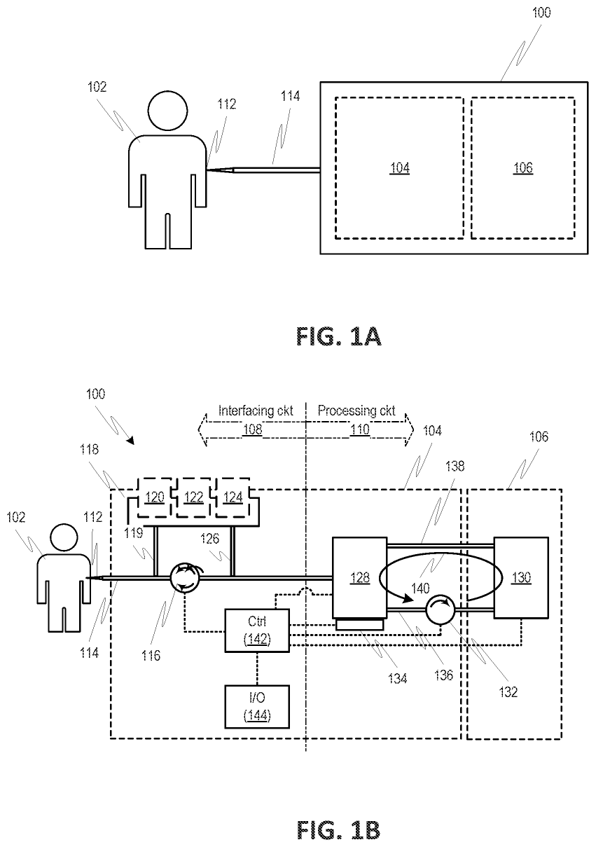

FIG. 1A illustrates aspects of a generalized blood treatment system 100 that employs batch processing and a legacy extracorporeal system. The system 100 can include a primary module 104 and a legacy treatment module 106. The primary module 104 can be designed to transfer blood to/from patient 102 and hold blood for processing. For example, a vascular access 112 is coupled to a single-lumen I/O conduit 114 to provide blood from patient 102 to primary module 104 for processing. The vascular access 112 can comprise a needle, catheter, or any other device for connecting to the patient vascular system known in the art. The legacy treatment module 106 can be designed to affect a treatment on blood passing thereto, for example, a dialysis treatment including, but not limited to, a hemodialysis (HD) system, a hemoperfusion system, hemofiltration system, hemodiafiltration system, a liver dialysis system, an oxygenator, an extracorporeal CO₂ removal (ECCO₂R) system or a combination thereof, including devices with combined function, e.g., a combination dialysis filter/hemoperfusion filter. For example, the legacy hemodialysis (HD) system can be a portable system or a system suitable for home use (HHD).

The legacy system can be a combination of legacy systems, such as, for example, hemodialysis (HD) and hemoperfusion, or liver dialysis and hemoperfusion, or extracorporeal CO₂ removal and hemodialysis (HD) or a combination of hemoperfusion, hemodialysis (HD) and ECCO₂R. Others include oxygenators plus any other blood processing/filtration systems.

Legacy devices include the NxStage Versi™ HD by Fresenius Medical Care Holdings, Inc. Other systems include the Tablo® system by Outset Medical, and Prismax™ by Baxter, which allows CRRT (hemodialysis) using a ST 150 filter by Oxiris. Other legacy devices include Omni by Braun, which performs continuous blood purification treatments and therapeutic plasma exchange with removal of plasma components. Another legacy instrument is the DIMI from Dialco, which is indicated to treat patients with acute and/or chronic renal failure with or without fluid overload using hemodialysis (“HD”), hemodiafiltration (“HDF”), hemofiltration (“HF”) and/or ultrafiltration (“UF”) in hospital, clinical settings or at home. Those of skill in the art will know of other legacy systems useful in the present disclosure. Further systems include those by CVS home dialysis devices, and Dialty.

Other legacy devices include plasma exchange and apheresis devices. For example, a membrane therapeutic plasma exchange (mTPE) is performed with a highly permeable filter and dialysis equipment. In certain instances, plasma exchange is achieved with simultaneous infusion of a replacement solution. Plasma is removed and pumped through the large-pore membrane of a legacy plasma filter, while a colloid solution, such as albumin and/or plasma, or a combination of crystalloid/colloid solution, is infused post-plasma filter to replace the removed plasma.

In certain aspects, apheresis allows for the collection of specific blood components which, depending on the patient's condition, are replaced with similar components received from blood donors, removed and stored for later use, or discarded. Using apheresis, blood is temporarily removed from the vein and put through an apheresis machine which separates the blood. For example, red blood cell exchange is the process where a patient's red blood cells are removed and replaced with donor red blood cells. Red blood cell exchange can be used in the treatment of sickle cell disease.

In some embodiments, module 104 is configured to connect to module 106 by appropriate blood-compatible connectors. For example, the primary module 104 may be a standalone system with releasable connectors or interface that allows an installed or legacy treatment module 106 performing a blood treatment with dual lumens to be connected to patient 102 with a single lumen 112. Alternatively, or additionally, connecting module 104 to legacy treatment module 106 may be effective to renew or enhance a treatment component (e.g., an HPF device) expended in the blood processing. Thus, the system 100 may offer different blood or body fluid treatments by simply connecting to a legacy treatment module 106.

Turning to FIG. 1B, in some embodiments, system 100 may be considered to have an interfacing circuit 108 that conveys blood to/from patient 102 and a processing circuit 110 that treats the blood. For example, the interfacing circuit 108 may be constituted by or comprises components fully or substantially contained in primary module 104, while the processing circuit 110 may be constituted by some components contained in primary module 104 and other components contained in legacy treatment module 106. The interfacing circuit 108 can include, for example, single lumen I/O conduit 114, and a first blood pump 116 and a fluid/drug module 118 with associated supply conduits 119, 126. The first pump 116 can be a Harvard-type apparatus or syringe pump or infuse/withdraw pump. The processing circuit 110 can include, for example, a blood reservoir or chamber 128, a second blood pump 132, a legacy or installed treatment device 130 (e.g., filtration device), and conduits 136, 138 that form a recirculation fluid circuit 140 between the reservoir 128 and legacy treatment device 130. The second pump 132 can be a Harvard-type apparatus or syringe pump or infuse/withdraw pump.

In certain aspects, primary module 104 refreshes legacy treatment module 106. Primary module 104 may include new functionalities not originally present in legacy systems such as digital communication for off-site monitoring and telemedicine.

In some embodiments, system 100 may also include a controller 142 operatively coupled to the various components of the interfacing 108 and processing 110 circuits for controlling operation thereof to effect batch processing and blood treatment. System 100 may also include an input/output (I/O) module 144, which can be operatively coupled to the controller 142. In some embodiments, the I/O module 144 can be configured to convey control signals, data, or any other information to external systems, for example, to coordinate operation of system 100 with legacy or installed treatment devices or to convey a status of treatment to a local or remote monitoring system. By including the new components and functionalities in primary module 106, legacy systems can be refreshed, reused and recycled with new up-to-date functionalities. Alternatively, or additionally, the I/O module 144 can receive operating instructions from and/or provide information (e.g., visual or auditory) to a medical operator of the system 100 or the patient 102.

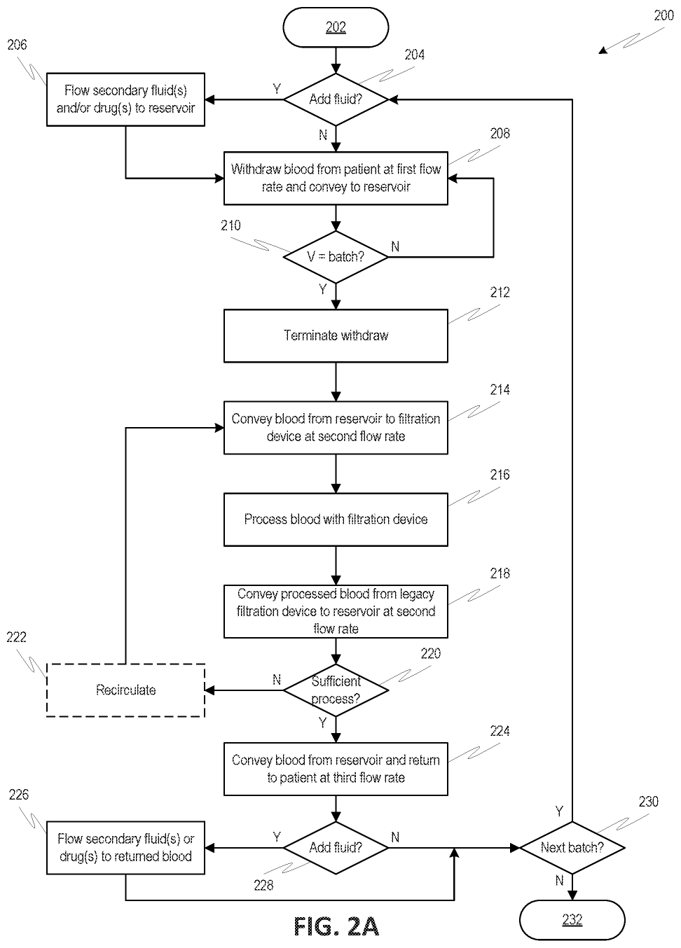

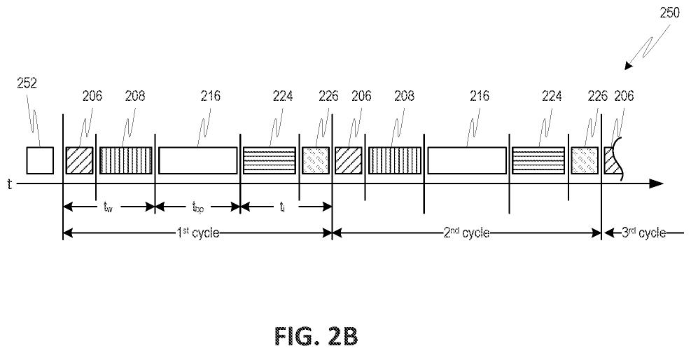

Referring to FIGS. 1B and 2A, an exemplary process 200 for operation of system 100 will be described. The process 200 can initiate at 202 and proceed to 204, where it is determined if a secondary fluid or drug is to be added to the blood reservoir 128. For example, controller 142 can determine if secondary fluid addition is required based on the type of legacy treatment module 106, the type of blood treatment to be performed, and/or custom instructions received via I/O 144. For example, when legacy treatment module 106 provides HDF, controller 142 can instruct the addition of hemofiltration or replacement fluid. Alternatively, or additionally, the controller 142 can instruct the addition of a drug or a therapeutic agent. For example, when the patient has not otherwise been dosed with an anticoagulant, controller 142 can instruct the addition of an appropriate anticoagulant, such as, but not limited to, heparin, citrate-based anticoagulants, nafamostat, or epoprostenol.

If it is determined at 204 that secondary fluid and/or drug is to be added, the process 200 can proceed to 206, where the secondary fluid and/or drug is flowed from secondary fluid supply 120 and/or anticoagulant supply 122 in fluid/drug module 118 to the blood reservoir 128. For example, controller 142 can control fluid/drug module 118, first pump 116, and various valves or other fluid control components (not shown) to pump secondary fluid and/or anticoagulant from module 118 via one or more input conduits 119 to single-lumen conduit 114, and then on to blood reservoir 128.

Once sufficient secondary fluid and/or drug has been provided to reservoir 128, or when it is otherwise determined at 204 that secondary fluid or drugs are not needed, the process 200 can proceed to 208, where blood is withdrawn from patient 102 via access 112 and conveyed to reservoir 128 for temporary storage until treatment processing. For example, controller 142 can control first pump 116 and various valves or other fluid control components (not shown) to pump the blood from patient 102 along single-lumen conduit 114 to the reservoir 128 at a first flow rate. The blood conveying 208 can continue via 210 until a predetermined blood volume (V) is obtained in the reservoir 128. The predetermined blood volume may be adjustable based on a size of patient 102, for example, 2-7% or 1-15% such as 1, 2, 3, 4, 5, 6, 7, 8, 9, 10, 11, 12, 13, 14 or 15% of a total blood volume of patient 102. For example, the predetermined blood volume may be 10-300 ml, or 10 ml to 1000 ml and may be set by the patient 102 or system operator via I/O module 144.

The controller 142 can monitor the volume of blood in reservoir 128 and determine at 210 whether the predetermined blood volume has been met. For example, the weight of reservoir 128 and contents therein can be monitored by an accurate weight sensor 134, e.g., a gravity scale. Because the blood volume in reservoir 128 is relatively small (e.g., less than 300 ml), the reservoir 128 should be weighed very accurately to avoid incorrect volume correlations. For example, the weight sensor may have an accuracy down to 1 gram or less. Those of skill in the art will know of other sensors to measure fluid level including, but not limited to, floats, gauges, capacitive level sensors, light sensors and other volume or weight sensors, which can be used.

Controller 142 can then correlate changes in weight of reservoir 128 to changes in fluid/blood volume therein. Controller 142 can also correlate changes or the presence of a signal when other volume levels sensors are used. In some embodiments, weight sensor 134 provides signals to controller 142 in real-time during fill of reservoir 128. The sensor 134 and/or controller 142 may thus be configured to compensate for any weight fluctuations due to fluid dynamics/vibration within the reservoir during the blood flow 208. Alternatively or additionally, controller 142 may sample signals from the weight sensor 134 and determine at 210 if sufficient volume has been achieved during intermittent pauses in flow 208 to allow blood in reservoir 128 to settle.

Although 208-210 is shown as occurring after 204-206, it is also possible in some embodiments that the order may be reversed, i.e., such that blood is withdrawn from patient 102 and stored in reservoir 128 before the addition of secondary fluid and/or anticoagulant to the reservoir 128. Moreover, in some embodiments, fluid conveyances other than pump 116 can be used for the secondary fluid or anticoagulant. For example, input conduit 119 of fluid/drug module 118 may bypass single-lumen conduit 114 and interface directly with the blood reservoir 128. A fluid conveyance (not shown) arranged between the fluid/drug module 118 and the reservoir 128 can transport the secondary fluid or anticoagulant to reservoir 128, such that secondary fluid/drug flow 206 may be able to occur simultaneously with supply 208 of blood to the reservoir 128. The fluid conveyance may be a fluid pump similar to pump 116, a Harvard-type apparatus, a syringe pump, a gravity-feed controlled by an appropriate valve, or any other device known in the art.

Once the predetermined blood volume in reservoir 128 has been reached at 210, the process 200 can proceed to 212, where withdrawal of blood from patient 102 is terminated for that cycle. For example, controller 142 can control first pump 116 and various valves or other fluid control components (not shown) to stop the blood flow from patient 102 and to otherwise isolate single-lumen conduit 114 from blood reservoir 128 for subsequent treatment processing.

The process 200 can thus proceed to 214, where blood treatment processing may be initiated. In particular, blood from reservoir 128 (potentially with secondary fluid and/or anticoagulant) is conveyed at 214 to legacy filtration device 130, where the blood is subjected to a legacy treatment process at 216 (e.g., flowing through to affect a dialysis treatment), and then returned to the reservoir 128 at 218. For example, controller 142 can control second pump 132 and various valves or other fluid control components (not shown) to flow blood from reservoir 128 along conduit 136, through legacy filtration device 130, and back to reservoir 128 via conduit 138. The flowing of blood in each of steps 214-218 may be at a second flow rate. In general, the second flow rate is greater than the first flow rate (used to withdraw blood from patient 102) to enhance solute clearance efficiency. For example, the second flow rate can be 50-500 ml/min and may be at least 1.25 times, and preferably at least 2 times, greater than the first flow rate.

In other words, the first and or third flow rate is about 5 ml/min to about 250 ml/min, or about 5 ml/min, 10 ml/min, 15 ml/min, 20 ml/min, 25 ml/min, 30 ml/min, 35 ml/min, 40 ml/min, 45 ml/min, 50 ml/min, 55 ml/min, 60 ml/min, 65 ml/min, 70 ml/min, 75 ml/min, 80 ml/min, 85 ml/min, 90 ml/min, 95 ml/min, 100 ml/min, 105 ml/min, 110 ml/min, 115 ml/min, 120 ml/min, 125 ml/min, 130 ml/min, 135 ml/min, 140 ml/min, 145 ml/min, 150 ml/min, 155 ml/min, 160 ml/min, 165 ml/min, 170 ml/min, 175 ml/min, 180 ml/min, 185 ml/min, 190 ml/min, 195 ml/min, 200 ml/min, 205 ml/min, 210 ml/min, 215 ml/min, 220 ml/min, 225 ml/min, 230 ml/min, 235 ml/min, 240 ml/min, 245 ml/min, and/or 250 ml/min.

The second flow rate is at least 1.25 times, and preferably at least 2 times, greater than the first flow rate or 50-750 ml/min, or about 50 ml/min, 55 ml/min, 60 ml/min, 65 ml/min, 70 ml/min, 75 ml/min, 80 ml/min, 85 ml/min, 90 ml/min, 95 ml/min, 100 ml/min, 105 ml/min, 110 ml/min, 115 ml/min, 120 ml/min, 125 ml/min, 130 ml/min, 135 ml/min, 140 ml/min, 145 ml/min, 150 ml/min, 155 ml/min, 160 ml/min, 165 ml/min, 170 ml/min, 175 ml/min, 180 ml/min, 185 ml/min, 190 ml/min, 195 ml/min, 200 ml/min, 205 ml/min, 210 ml/min, 215 ml/min, 220 ml/min, 225 ml/min, 230 ml/min, 235 ml/min, 240 ml/min, 245 ml/min, 250 ml/min, 255 ml/min, 260 ml/min, 265 ml/min, 270 ml/min, 275 ml/min, 280 ml/min, 285 ml/min, 290 ml/min, 295 ml/min, 300 ml/min, 305 ml/min, 310 ml/min, 315 ml/min, 320 ml/min, 325 ml/min, 330 ml/min, 335 ml/min, 340 ml/min, 345 ml/min, 350 ml/min, 355 ml/min, 360 ml/min, 365 ml/min, 370 ml/min, 375 ml/min, 380 ml/min, 385 ml/min, 390 ml/min, 395 ml/min, 400 ml/min, 405 ml/min, 410 ml/min, 415 ml/min, 420 ml/min, 425 ml/min, 430 ml/min, 435 ml/min, 440 ml/min, 445 ml/min, 450 ml/min, 455 ml/min, 460 ml/min, 465 ml/min, 470 ml/min, 475 ml/min, 480 ml/min, 485 ml/min, 490 ml/min, 495 ml/min, 500 ml/min, 505 ml/min, 510 ml/min, 515 ml/min, 520 ml/min, 525 ml/min, 530 ml/min, 535 ml/min, 540 ml/min, 545 ml/min, 550 ml/min, 555 ml/min, 560 ml/min, 565 ml/min, 570 ml/min, 575 ml/min, 580 ml/min, 585 ml/min, 590 ml/min, 595 ml/min, 600 ml/min, 605 ml/min, 610 ml/min, 615 ml/min, 620 ml/min, 625 ml/min, 630 ml/min, 635 ml/min, 640 ml/min, 645 ml/min, 650 ml/min, 655 ml/min, 660 ml/min, 665 ml/min, 670 ml/min, 675 ml/min, 680 ml/min, 685 ml/min, 690 ml/min, 695 ml/min, 700 ml/min, 705 ml/min, 710 ml/min, 715 ml/min, 720 ml/min, 725 ml/min, 730 ml/min, 735 ml/min, 740 ml/min, 745 ml/min, and/or 750 ml/min.

At 220, it can be determined if the blood in reservoir 128 has been subjected to sufficient treatment processing by 214-218. For example, controller 142 can determine whether sufficient treatment has occurred based on an elapsed time of the processing, a magnitude of the second flow rate, and/or a volume of the blood batch in reservoir 128. If sufficient processing has not been achieved at 220, the process 200 can proceed to 222, where the blood is optionally recirculated and reprocessed by returning to 214. Thus, in embodiments, the flowing of blood along recirculation circuit 140 in 214-218 can be repeated such that each portion of the blood passes through filtration device 130 more than twice (e.g., 2-10 times), and preferably several times in an iterative process. For example, the recirculation of blood may be such that the entire volume of the reservoir passes through the filtration device at least three times before being returned to the patient. The repeated processing of the same blood by the filtration device may achieve further improved clearance efficiency as compared to conventional single-pass RRT systems.

Alternatively or additionally, controller 142 can correlate changes in weight of reservoir 128 (or other fluid level sensor as measured by sensor 134) to a stage of treatment processing. For example, an amount of fluid removed from the blood by the filtration device 130 can correlate with a stage of the treatment, which fluid removal can be detected in changes in instantaneous or average weight or level of fluid of reservoir 128 and contents therein. Thus, in some embodiments, weight sensor 134 provides signals to controller 142 in real-time during flow of blood from/to reservoir 128. The sensor 134 and/or controller 142 may thus be configured to compensate for any weight fluctuations due to fluid dynamics/agitation within the reservoir during the blood flows 214-218. Alternatively, or additionally, controller 142 may sample signals from the weight sensor 134 and determine at 220 if sufficient processing has been achieved during intermittent pauses in blood flows 214-218 to allow blood in reservoir 128 to settle.

Although shown as separate sequential steps in FIG. 2A, in practice 214-222 may occur simultaneously, with blood recirculating between reservoir 128 and filtration device 130 continuously until sufficient processing has been achieved at 220. In some embodiments, the continuous recirculation may be periodically interrupted, for example, to allow for a more accurate weight measurement or fluid volume level of blood or body fluid reservoir 128 by sensor 134.

Once sufficient treatment processing of blood in reservoir 128 has been reached at 220, the process can proceed to 224, where the recirculation 222 is terminated and treated blood in reservoir 128 is returned to patient 102 via access 112. For example, controller 142 can control first pump 116 and various valves or other fluid control components (not shown) to pump the blood from reservoir along single-lumen conduit 114 to the access 112 at a third flow rate. Since the blood return uses the same conduit 114 and access 112 as the blood withdrawal, the third flow rate can be, but does not need to be, the same as the first flow rate.

The process 200 can then proceed to 228, where it is determined if a secondary fluid or drug is to be added to patient 102. For example, when the patient was previously dosed with anticoagulant at 206, controller 142 can instruct the addition of an appropriate anticoagulant reversal agent, such as, but not limited to protamine and/or calcium. Alternatively, or additionally, controller 142 can determine if secondary fluid addition to patient 102 is required based on the type of legacy treatment module 106, the type of blood treatment performed, and/or custom instructions received via I/O 144. For example, controller 142 can instruct the infusion of a volume of replacement fluid such as albumin to patient 102. Alternatively, or additionally, the controller 142 can determine at 228 to use secondary fluid (e.g., buffer or saline) to flush conduit 114 and access 112 in preparation for a subsequent batch at 230.

If it is determined at 228 that secondary fluid and/or drug is to be added, the process 200 can proceed to 226, where the secondary fluid and/or drug is flowed from secondary fluid supply 120 and/or anticoagulant reversal supply 124 in fluid/drug module 118 to the patient 102. For example, controller 142 can control fluid/drug module 118, first pump 116, and various valves or other fluid control components (not shown) to pump secondary fluid and/or anticoagulant reversal agent from module 118 via one or more input conduits 126 to single-lumen conduit 114, and then on to patient 102.

Once sufficient secondary fluid and/or drug has been provided to patient 102, or when it is otherwise determined at 228 that secondary fluid or drugs are not needed, the process 200 can proceed to 230, where it is determined if another batch of blood for the same patient 102 should be processed. For example, controller 142 can control system 100 to repeat process 200 for multiple sequential batches until an entire blood volume of the patient 102 has been processed (e.g., 4-6 liters of blood). Alternatively, or additionally, controller 142 can control system 100 to repeat process 200 until a predetermined time limit or predetermined number of repetitions or volume of body fluid has been reached. I/O module 144 can be used by the patient 102 or operator to set the predetermined time limit or number of repetitions or volume of body fluid. If further batches are desired at 230, the process 200 returns to 204. Otherwise, the process 200 may terminate at 232 until initiated again for the same patient 102 or a different patient.

FIG. 2B shows a time map 250 corresponding to the process 200 of FIG. 2A. The overall treatment process may begin with an initial setup 252, where system 100 is connected to patient 102. For example, a needle serving as vascular access 112 can be placed into the vascular system of the patient 102 and the needle connected to single-lumen conduit 114 of system 100. Alternatively, a previously installed catheter serving as vascular access 112 can be coupled to single-lumen conduit 114 of system 100. After appropriate setup 252, a blood batch processing cycle is performed and can be sequentially repeated on additional batches in a continuous manner or until a termination condition is met, for example, until an entire blood volume of the patient has been processed. Each blood batch processing cycle comprises a batch preparation stage (constituted by secondary fluid/drug flow 206 and blood withdrawal 208), a blood treatment stage (constituted by blood treatment 216), and a batch return stage (constituted by blood infusion 224 and secondary fluid/drug flow 226).

The batch preparation and batch return stages can employ fluid flow rates less than that of blood treatment stage. In some embodiments, the batch preparation stage and batch return stage employ fluid flow rates that are substantially the same. As such, a time (t_{w}) for the batch preparation stage and a time (tᵢ) for the batch return stage may also be substantially the same. These times may be based on a volume of the blood batch, sizes of the vascular access 112 and single-lumen conduit 114, and fluid flow rate, among other things or metrics. A time (t_{bp}) for the blood treatment stage may be similar to that of the other stages despite the higher fluid flow rate. Alternatively, the time (t_{bp}) for the blood treatment stage may be greater than that for either or both of the other stages. The blood treatment stage time (t_{bp}) may be based on a volume of the blood batch, type of filtration device, fluid flow rate, and desired degree of recirculation (e.g., number of passes of blood through the filtration device), among other things or metrics.

In some embodiments, the time for each cycle is designed to be less than 10 minutes. For example, the total time for each cycle may be 4-7 minutes, thereby enabling up to 15 cycles to be achieved in an hour. When using a batch volume of around 200 ml, such cycle times may achieve blood processing levels comparable to conventional RRT systems. For example, t_{bp} may be around 3 minutes, with the remainder of the cycle time split equally between the remaining stages (e.g., t_{w}=tᵢ=˜3.5 minutes).

System 100 and/or process 200 (and/or any of the subsequently discussed embodiments) can be adapted to provide various dialysis treatment therapies, including continuous RRT, periodic intermittent RRT, nocturnal dialysis, daily home dialysis, or any other dialysis or blood purification application. The use of batch processing by system 100 and/or process 200 advantageously allows a single-lumen conduit 114 to be used for both withdrawal of blood from patient 102 and later infusion of processed blood to patient 102, unlike conventional RRT systems where two lumens are used to simultaneously withdraw blood from and infuse processed blood to the patient. This single access point or port can ease the burden of vascular access in both acute and chronic patients.

Moreover, the decoupling of flow rates allows for a smaller size vascular access 112 than would otherwise be required to support the second flow rate through legacy filtration device 130. Thus, system 100 may employ needles or catheters having a size less than that typically used in conventional RRT systems, which smaller size (and reduced number) may be better tolerated (or at least less painful or intrusive) by patient 102. The decoupling of flow rates also allows a higher second flow rate to be used than would otherwise be possible with conventional RRT systems, thereby improving clearance, especially of middle molecules (e.g., 500 Daltons to 60 kD). In some cases, it may be advantageous to use a slower flow rate through the ‘filter’ than the withdrawal rate from the patients, e.g., if the efficiency of the filter requires a long residence time.

In general, middle-molecule clearance can be achieved using (1) a high-flux dialyzer, (2) high blood flow rates, and (3) high dialysate flow rates, the combination of which is difficult to achieve in conventional RRT systems but is readily provided by system 100. Middle-molecule clearance can be measured by a representative middle molecule such beta 2 microglobulin. For system 100 and/or process 200, middle molecule clearance as measured by beta 2 microglobulin of at least 25 ml/min, and preferably 80-130 ml/min, can be achieved. For example, system 100 and/or process 200 can achieve a middle molecule clearance as measured by beta 2 microglobulin clearance greater than 100 ml/min with a single-lumen access 114, e.g., a catheter smaller than 7 French or a needle smaller than 17 gauge.

In certain instances, the catheter or needle is about 2 to 20 French such as 2, 3, 4, 5, 6, 7, 8, 9, 10, 11, 12, 13, 14, 15, 16, 127, 18, 19, 20 French, or 10-23 gauge such as 10, 11, 12, 13, 14 15, 16, 17, 18, 19, 20, 21, 22, 23-gauge needle.

System 100 and/or process 200 (and/or any of the subsequently discussed embodiments) may further exhibit one or more of the following advantages:

In certain aspects, blood batches can be small (e.g., ≤300 ml) and anticoagulated, and therefore a smaller capacity filtration device (e.g., hemofilter) can be utilized for the treatment processing. The smaller components may reduce system costs.

In certain aspects, the smaller filtration device coupled with relatively small batch volume can yield a footprint and/or three-dimensional size that is less than conventional RRT systems. The overall extracorporeal blood treatment system may thus be substantially portable, or at least more so than conventional RRT systems.

In certain aspects, blood batches can be small, and therefore an effective amount of anticoagulant may be used that is less than that required for conventional RRT systems.

In certain aspects, the anticoagulant may be localized (e.g., within system 100 and at the infusion site in the patient) rather than being distributed through the vascular system, which may avoid patient complications. Any anticoagulant infused into the patient may also be reversed by delivery of an anticoagulant reversal agent by the system. The use of blood-contacting components of the circuit that are surface modified to improve blood compatibility may, in some cases, obviate the need for systemic or local anticoagulation. Examples include covalently bound heparin, surface modifiers added to the polymer before the components are fabricated and/or polymers that are inherently blood compatible due to their bulk composition or surface-active end groups incorporated during synthesis.

In certain aspects, blood is only processed in batches, and therefore the risk of a blood leak in processing circuit 110 causing significant blood loss is mitigated. Moreover, since the first flow rate for blood withdrawal is relatively slower, the risk of significant blood loss due to a blood leak in the interfacing circuit 108 is also reduced.

In certain aspects, since the dual-lumen catheter of conventional RRT systems is not required in the disclosed systems, inefficiencies due to blood recirculation can be avoided.

In certain aspects, batch size, flow rates, and/or processing time can all be customized, for example, to take into account patient size or illness severity. Smaller withdrawal volumes of blood may decrease hemodynamic instabilities often seen when a conventional RRT session is initiated.

In some embodiments, the methods and systems disclosed can be used to process other body fluids. For example, accumulation of fluid in the abdominal cavity is called ascites. Ascites can be common with patients with cirrhosis, liver disease or congestive heart failure. When removing a body fluid such as ascites, a diuretic can also be administered. Commonly used diuretics include spironolactone (Aldactone) and/or furosemide (Lasix). When fluid accumulation cannot be treated optimally with diuretics and a salt restricted diet, patients may require a large amount of fluid be removed (paracentesis) for relief of symptoms. The disclosure includes methods and systems for treating ascites, by the withdrawal of ascites. Optionally, the withdrawn ascitic fluid can be concentrated and reinfused.

Paracentesis is carried out under strict sterile conditions. Ascites is withdrawn from patient 102 via access 112 and conveyed to reservoir 128 for temporary storage until treatment processing. Pump 116 can be used to remove the ascitic fluid at a flow rate of from about 50 ml/min to about 200 ml/min such as about 100 ml/min to about 150 ml/min. Alternatively, ascitic fluid removal may use gravity. The needle is usually inserted into the left or right lower abdomen, where the needle is advanced through the subcutaneous tissue and then through the peritoneal cavity. In certain aspects, the ascitic fluid is drained in a single session, assisted by gentle mobilization of the cannula or turning patient 102 if necessary.

The body fluid (e.g., ascites) from reservoir 128 is conveyed to filtration device 130, where the ascites is subjected to a treatment process such as concentration and is thereafter returned to the reservoir 128. The concentrated ascites (e.g., a protein rich concentrate) can be returned to patent 102 via conduit 114. Albumin may also be infused in lieu of the concentrated ascites, or in addition to the concentrated ascites. Another example of treating a body fluid other than blood is the treatment of spinal fluid in meningitis. Very slow withdrawal and small batch size are required, whereas the flow rate through a hemoperfusion device capable of removing pathogens (e.g., the Seraph® 100 Microbind® Affitity Blood Filter, ExThera Medical Corporation, Martinez, CA) can be performed at a higher flow rate to affect more passes per unit time through the filter.

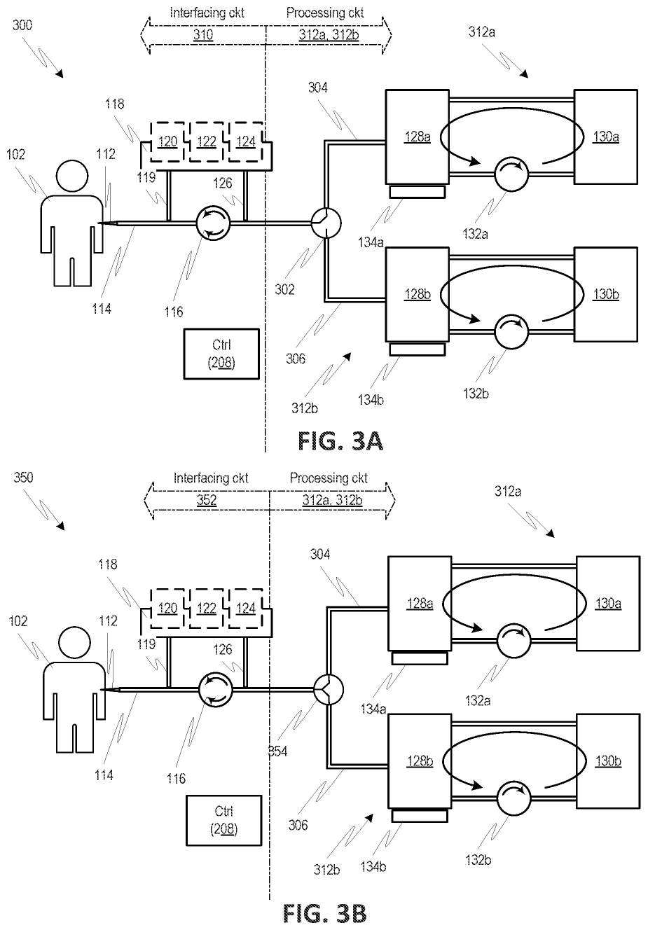

Although the description above has focused on the use of single blood reservoir 128, embodiments of the disclosed subject matter are not limited thereto. For example, the legacy system can be a combination of legacy systems, such as, for example, hemodialysis (HD) and hemoperfusion, or liver dialysis and hemoperfusion, or extracorporeal CO₂ removal and hemodialysis (HD) or a combination of hemoperfusion, hemodialysis (HD) and ECCO₂R. In certain aspects, the extracorporeal blood treatment systems and methods can utilize more than one blood reservoir for serial or parallel treatment processing. These may include one or more legacy treatment systems. For example, FIG. 3A shows a simplified layout for a generic extracorporeal blood treatment system 300 utilizing a pair of blood reservoirs 128a, 128b providing serial blood treatment processing. System 300 includes an interfacing circuit 310 and a pair of processing circuits 312a, 312b. Each processing circuit 312a, 312b can have respective blood reservoirs 128a, 128b, weight or fluid level sensors 134a, 134b, blood pumps 132a, 132b, which may be Harvard-type apparatuses and filtration devices 130a, 130b. Each processing circuit 312a, 312b is thus substantially similar to processing circuit 110 of FIG. 1B and may operate independently of each other to affect a blood treatment in a similar manner to processing circuit 110.

The interfacing circuit 310 is substantially similar to interfacing circuit 108 of FIG. 1B and thus may operate in a similar manner to interfacing circuit 108. However, interfacing circuit 310 further includes a fluid switch 302 (or combination of valves or other flow control devices to provide the effect of a switch) that connects single lumen conduit 114 to either an inlet conduit 304 of first processing circuit 312a or an inlet conduit 306 of second processing circuit 312b. Since only one processing circuit 312a, 312b can be connected to single lumen conduit 114 by switch 302 at a time, processing circuits 312a, 312b may be considered to operate serially.

For example, in FIG. 3A switch 302 selects for processing circuit 312a, such that blood or body fluid from patient 102 can be conveyed to reservoir 128a or processed blood from reservoir 128a can be returned to patient 102 via single lumen conduit 114 and inlet conduit 304. Meanwhile, processing circuit 312b is de-selected by switch 302. While de-selected, processing circuit 312b may recirculate previously withdrawn blood between reservoir 128b and filtration device 130b to affect a blood treatment. Thus, blood treatment processing by one of the processing circuits 312a, 312b may occur while the other of the processing circuits 312a, 312b is withdrawing or infusing blood, thereby taking advantage of what would otherwise be considered blood processing downtime in a single blood reservoir system. Alternatively, processing circuit 312b may be idle during the de-selected period. Similar to control system 142, control system 308 controls operation of components of the interfacing circuit 310 (for example, selection by switch 302) and processing circuits 312a, 312b. One of skill in the art will recognize that one or more additional processing circuit(s) are possible such as 312c, 312d, etc., by including additional switches.

In another example, FIG. 3B shows a simplified layout for a generic extracorporeal blood treatment system 350 utilizing a pair of blood reservoirs 128a, 128b providing parallel blood treatment processing. System 350 includes an interfacing circuit 352 and a pair of processing circuits 312a, 312b, each of which is substantially similar to processing circuit 110 of FIG. 1B and may operate independent of each other to affect a blood treatment in a similar manner to processing circuit 110.

The interfacing circuit 352 is similar to interfacing circuit 310 of FIG. 3A but includes a fluidic union 354 (or combination of valves or other flow control devices to provide the effect of a union) instead of a switch 302. The union 354 (e.g., a Y-connector) connects single lumen conduit 114 to both the inlet conduit 304 of first processing circuit 312a or the inlet conduit 306 of second processing circuit 312b. Since both processing circuits 312a, 312b are connected to single lumen conduit 114 by union 354 at a time, processing circuits 312a, 312b may be considered to operate in parallel. One of skill in the art will recognize that one or more additional processing circuit(s) are possible such as 312c, 312d, etc., by including additional unions.

For example, blood from patient 102 can be simultaneously conveyed to reservoirs 128a, 128b or processed blood from reservoirs 128a, 128b can be simultaneously returned to patient 102 via single lumen conduit 114 and inlet conduits 304, 306. As such, the blood volume from the patient 102 traveling along single lumen conduit 114 can be split between each of the blood reservoirs 128a, 128b, and blood returning from reservoirs 128a, 128b can be combined prior to introduction to patient at vascular access 112. Processing circuits 312a, 312b may also recirculate blood between reservoirs 128a, 128b and filtration devices 130a, 130b at the same time to effect a parallel blood treatment. Similar to control system 142, control system 308 controls operation of components of interfacing circuit 352 and processing circuits 312a, 312b.

Although processing circuits 312a, 312b are illustrated as being identical in FIGS. 3A-3B, in some embodiments, filtration devices 130a, 130b may be different (i.e., offering separate treatment modalities). For example, a first fraction of the withdrawn blood is subjected to a first treatment modality by processing circuit 312a while a second fraction of the withdrawn blood is subjected to a second treatment modality (which may be different or complementary to the first treatment modality or regimens) by processing circuit 312b. Moreover, although FIGS. 3A-3B illustrate exemplary systems with a pair of blood reservoirs, serial or parallel processing with additional blood reservoirs is also possible according to one or more contemplated embodiments. Indeed, the teachings of FIGS. 3A-3B can be readily extended to three or more blood reservoirs (and associated processing circuits) by appropriate design of switching (e.g., switch 302) or union (e.g., union 354) components. In some embodiments, a combination of serial and parallel processing circuits are contemplated.

In this application, unless specifically stated otherwise, the use of the singular includes the plural, and the separate use of “or” and “and” includes the other, i.e., “and/or.” Furthermore, use of the terms “including” or “having,” as well as other forms such as “includes,” “included,” “has,” or “had,” are intended to have the same effect as “comprising” and thus should not be understood as limiting.

Any range described herein will be understood to include the endpoints and all values between the endpoints. Whenever “substantially,” “approximately,” “essentially,” “near,” or similar language is used in combination with a specific value, variations up to and including 10% of that value are intended, unless explicitly stated otherwise.

It is thus apparent that there is provided, in accordance with the present disclosure, syringe-based manual extracorporeal blood treatment systems and methods employing batch processing. Many alternatives, modifications, and variations are enabled by the present disclosure. While specific examples have been shown and described in detail to illustrate the application of the principles of the present invention, it will be understood that the invention may be embodied otherwise without departing from such principles. For example, disclosed features may be combined, rearranged, omitted, etc. to produce additional embodiments, while certain disclosed features may sometimes be used to advantage without a corresponding use of other features. Accordingly, Applicant intends to embrace all such alternative, modifications, equivalents, and variations that are within the spirit and scope of the present invention.

What is claimed is:

1. A blood treatment method, the method comprising:
(a) conveying a volume of blood via a first conduit from a vascular access of a patient to a blood chamber at a first flow rate, the first conduit having only a single lumen;
(b) conveying the blood from the blood chamber through a filtration device at a second flow rate, wherein the filtration device is part of a legacy system, to perform an extracorporeal treatment on the blood and returning the treated blood to the blood chamber; and
(c) returning the blood from the blood chamber to the vascular access of the patient at a third flow rate via the first conduit, wherein the second flow rate is decoupled from, and independent from of both the first and third flow rates.

2. The method of claim 1, wherein the first conduit is a needle or cannula forming at least part of the vascular access.

3. The method of claim 2, wherein the catheter or needle of the first conduit has a size of either 2 to 20 French or 10 to 23 gauge.

4. The method of claim 1, wherein the legacy system is a hemodialysis (HD) system, a hemoperfusion system, hemofiltration system, hemodiafiltration system, a liver dialysis system, an oxygenator, an extracorporeal CO₂ removal (ECCO₂R) system, a plasma exchange device and an apheresis device or a combination thereof.

5. The method of claim 4, wherein the legacy hemodialysis (HD) system is a portable system or suitable for home use (HHD).

6. The method of claim 4, wherein the combination of legacy systems is hemodialysis (HD) and hemoperfusion, liver dialysis and hemoperfusion, extracorporeal CO₂ removal and hemodialysis (HD) or hemoperfusion, hemodialysis (HD) and ECCO₂R.

7. The method of claim 4, wherein the combination of legacy systems is run in parallel or in series.

8. The method of claim 1, wherein the legacy system performs one or more mode(s) of hemodialysis selected from the group consisting of slow continuous ultrafiltration (SCUF), continuous venovenous hemodialysis (CVVHD), continuous venovenous hemodiafiltration (CVVHDF), and continuous venovenous high-flux hemodialysis (CVVHFD).

9. The method of claim 1, wherein the filtration device of the legacy system comprises a membrane or adsorption media.

10. The method of claim 9, wherein the membrane of the filtration device comprises at least one polymer which is a member selected from the group consisting of a polyamide, a polysulfone, a polyethersulfone, a cellulose acetate, a triacetate, a polyacrylonitrile and a polymethylmethacylate, each of the foregoing optionally surface modified for improved blood compatibility.

11. The method of claim 10, wherein the at least one polymer is a polysulfone.

12. The method of claim 1, wherein the second flow rate is 50 ml/min-500 ml/min, inclusive.

13. The method of claim 1, wherein the second flow rate is at least 300 ml/min.

14. A blood treatment system, the system comprising:
a reservoir for holding a batch of blood from a patient;
a first conduit for conveying blood from a vascular access of the patient during a first stage and for returning treated blood to the vascular access during a third stage, the first conduit having only a single lumen;
a legacy system for performing extracorporeal treatment on blood passing therethrough;
a recirculating blood processing loop connecting the reservoir to a legacy filter and an optional second filter device;
a blood pump for conveying blood in the recirculating blood processing loop; and
a controller configured to control the blood pump to repeatedly recirculate blood from the reservoir through the legacy filter during a second stage between the first and third stages.

15. The system of claim 14, wherein the legacy system is a hemodialysis (HD) system, a hemoperfusion system, hemofiltration system, hemodiafiltration system, a liver dialysis system, an extracorporeal CO₂ removal (ECCO₂R) system, a plasma exchange device and an apheresis device or a combination thereof.

16. The system of claim 15, wherein the legacy hemodialysis (HD) system is a portable system or suitable for home use (HHD).

17. The system of claim 15, wherein the combination of legacy systems is hemodialysis (HD) and hemoperfusion, liver dialysis and hemoperfusion, extracorporeal CO₂ removal and hemodialysis (HD) or hemoperfusion, hemodialysis (HD) and ECCO₂R.

18. The system of claim 14, wherein the first conduit is a needle or cannula forming at least part of the vascular access.

19. The system of claim 14, wherein the vascular access comprises multiple lumens, and the first conduit is coupled to respective outlets of the multiple lumens by a Y-connector.

20. The system of claim 14, wherein the reservoir has a volume of 10-300 ml, inclusive; or
wherein the controller controls the blood pump to generate a flow rate of blood in the processing fluid circuit or in the recirculating blood processing loop that is 50-500 ml/min, inclusive; or
wherein the controller controls the blood pump to generate a flow rate of blood in the processing fluid circuit or in the recirculating blood processing loop that is at least 300 ml/min; or
wherein the controller controls the blood pump, which is a first blood pump, to generate a first flow rate of blood in the processing fluid circuit and a second blood pump to generate a second flow rate of blood in the interfacing fluid circuit, and
the first flow rate is at least 1.25 times greater than the second flow rate.
